(19) United States
(12) Patent Application Publication
(10) Pub. No.: US 2021/0163677 A1
(43) Pub. Date: Jun. 3, 2021

(54) PHOTOACTIVE MATERIALS

(71) Applicant: Sumitomo Chemical Company Limited, Tokyo (JP)

(72) Inventors: Kiran Kamtekar, Godmanchester (GB); James Lee, Godmanchester (GB)

(73) Assignee: Sumitomo Chemical Company Limited, Tokyo (JP)

(21) Appl. No.: 17/105,288

(22) Filed: Nov. 25, 2020

(30) Foreign Application Priority Data
Nov. 29, 2019 (GB) 1917460.6

(51) Int. Cl. C08G 61/12; H01L 51/00

(52) U.S. Cl. CPC C08G 61/125; C08G 61/126; H01L 51/0036; H01L 51/0043; C08G 2261/3247; C08G 2261/12; C08G 2261/146; C08G 2261/3245; C08G 2261/3246; C08G 2261/314

(57) ABSTRACT

A material comprising a group of formula (I):

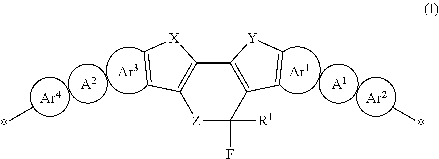

wherein: X and Y are each independently selected from S, O or Se; Z is O, S, NR² or CR³₂
Ar¹, A^{re}, Ar³ and Ar⁴ are each independently an unsubstituted or a substituted benzene, an unsubstituted or a substituted 5- or 6-membered heteroaromatic group or are absent;
A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent; R¹ is H or a substituent; R² is H or a substituent;
each R³ is independently H or a substituent; and * represents a point of attachment to a hydrogen or non-hydrogen group. The material may be used as an electron donor or an electron acceptor in an organic photoresponsive device.

### RELATED APPLICATIONS

This application claims foreign priority benefits under 35 U.S.C. § 119(a)-(d) or 35 U.S.C. § 365(b) of British application number GB 1917460.6, filed Nov. 29, 2019, the entirety of which is incorporated herein.

### BACKGROUND

Embodiments of the present disclosure relate to photoactive compounds and more specifically, but not by way of limitation, to photoactive materials containing electron-donating units suitable for us as an electron-donating material or an electron-accepting material in a photoresponsive device.

WO 2013/135339 discloses conjugated polymers containing divalent donor units is linked on both sides to an acceptor unit.

### SUMMARY

According to some embodiments of the present disclosure, there is provided a material comprising a group of formula (I):

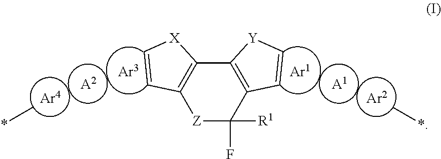

X and Y are each independently selected from S, O or Se;

Z is O, S, NR² or CR³₂;

Ar¹, Ar², Ar³ and Ar⁴ are each independently an unsubstituted or a substituted benzene, an unsubstituted or a substituted 5- or 6-membered heteroaromatic group or are absent;

A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent;

R¹ is H or a substituent;

R² is H or a substituent;

each R³ is independently H or a substituent; and

* represents a point of attachment to a hydrogen or non-hydrogen group.

Optionally, Ar¹, A¹, Ar², Ar³ A² and Ar⁴ are absent and the group of formula (I) has formula (Ic):

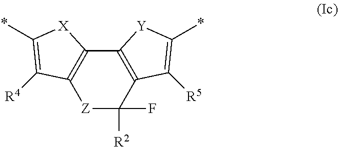

Optionally, the group of formula (I) is an electron donor group, the material further comprising at least one electron-accepting group bound directly to the group of formula (I).

According to some embodiments, the material is a polymer comprising a repeat unit of formula (Id):

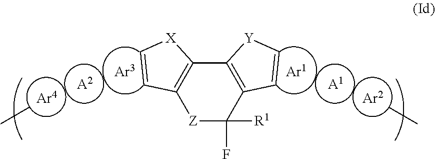

Optionally, the repeat unit of formula (Id) is selected from repeat units of formulae (Ie), (If) and (Ig):

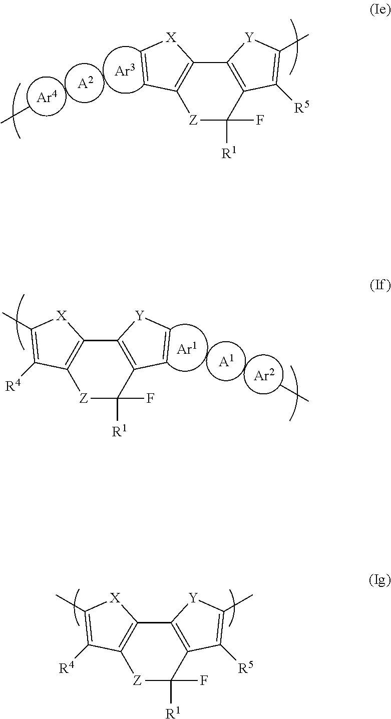

Optionally, the repeat unit of formula (I) is an electron-donating repeat unit and wherein the polymer further comprises an electron accepting co-repeat unit.

Optionally, the polymer comprises a repeating structure of formula:

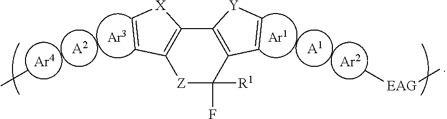

According to some embodiments of the present disclosure, there is provided a composition comprising an electron donor material and an electron acceptor material wherein the electron donor material is the material comprising a group of formula (I).

According to some embodiments of the present disclosure, there is provided a composition comprising an electron donor material and an electron acceptor material wherein the electron acceptor material is the material comprising a group of formula (I).

According to some embodiments of the present disclosure, there is provided a formulation comprising one or more solvents and a material or a composition as described herein wherein the material comprising the group of formula (I) is dissolved or dispersed in the one or more solvents.

According to some embodiments of the present disclosure, there is provided a photoresponsive device comprising an anode, a cathode and a photosensitive layer disposed between the anode and the cathode, wherein the photosensitive layer comprises a material or a composition as described herein.

Optionally, the photoresponsive device is an organic photodetector.

According to some embodiments of the present disclosure, there is provided a photosensor comprising a light source and a photoresponsive device as described herein, wherein the photoresponsive device is configured to detect light emitted from the light source.

Optionally, the light source emits light having a peak wavelength greater than 750 nm.

Optionally, the photosensor is configured to receive a sample in a light path between the organic photodetector and the light source.

According to some embodiments of the present disclosure, there is provided a method of forming an organic photoresponsive device as described herein. The method comprises formation of the photosensitive organic layer over one of the anode and cathode and formation of the other of the anode and cathode over the photosensitive organic layer.

Optionally, formation of the photosensitive organic layer comprises deposition of a formulation as described herein.

According to some embodiments of the present disclosure, there is provided a method of determining the presence and/or concentration of a target material in a sample, the method comprising illuminating the sample and measuring a response of a photoresponsive device as described herein.

### DESCRIPTION OF DRAWINGS

The disclosed technology and accompanying FIGURES describe some implementations of the disclosed technology.

FIG. 1 illustrates an organic photoresponsive device according to some embodiments.

The drawings are not drawn to scale and have various viewpoints and perspectives. The drawings are some implementations and examples. Additionally, some components and/or operations may be separated into different blocks or combined into a single block for the purposes of discussion of some of the embodiments of the disclosed technology. Moreover, while the technology is amenable to various modifications and alternative forms, specific embodiments have been shown by way of example in the drawings and are described in detail below. The intention, however, is not to limit the technology to the particular implementations described. On the contrary, the technology is intended to cover all modifications, equivalents, and alternatives falling within the scope of the technology as defined by the appended claims.

### DETAILED DESCRIPTION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Additionally, the words “herein,” “above,” “below,” and words of similar import, when used in this application, refer to this application as a whole and not to any particular portions of this application. Where the context permits, words in the Detailed Description using the singular or plural number may also include the plural or singular number respectively. The word “or,” in reference to a list of two or more items, covers all of the following interpretations of the word: any of the items in the list, all of the items in the list, and any combination of the items in the list. References to a layer “over” another layer when used in this application means that the layers may be in direct contact or one or more intervening layers are may be present. References to a layer “on” another layer when used in this application means that the layers are in direct contact. References to a specific atom include any isotope of that atom unless specifically stated otherwise.

The teachings of the technology provided herein can be applied to other systems, not necessarily the system described below. The elements and acts of the various examples described below can be combined to provide further implementations of the technology. Some alternative implementations of the technology may include not only additional elements to those implementations noted below, but also may include fewer elements.

These and other changes can be made to the technology in light of the following detailed description. While the description describes certain examples of the technology, and describes the best mode contemplated, no matter how detailed the description appears, the technology can be practiced in many ways. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms.

In the following description, for the purposes of explanation, numerous specific details are set forth in order to provide a thorough understanding of implementations of the disclosed technology. It will be apparent, however, to one skilled in the art that embodiments of the disclosed technology may be practiced without some of these specific details.

The present inventors have found that materials comprising a group of formula (I) may be used in a donor-acceptor system used in an organic photoresponsive device, e.g. a photovoltaic device such as a solar cell or an organic photodetector containing a bulk heterojunction layer containing a donor material and an acceptor material.

The materials may absorb long wavelengths of light, e.g. greater than about 750 nm, making them suitable for use in organic photodetectors for detection of light in the near-infrared range such as in the range of greater than about 750 nm or greater than about 1000 nm. The materials may absorb wavelengths of light that are between about 750 nm and about 2000 nm, between about 750 nm and about 1000 nm or between about 1000 nm to about 2000 nm.

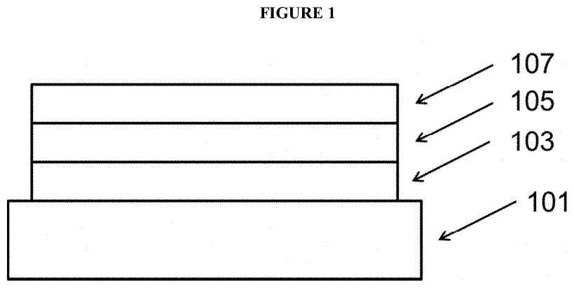

FIG. 1 illustrates an organic photoresponsive device according to some embodiments of the present disclosure. The organic photoresponsive device comprises a cathode 103, an anode 107 and a bulk heterojunction layer 105 disposed between the anode and the cathode. The organic photoresponsive device may be supported on a substrate 101, optionally a glass or plastic substrate.

At least one of the anode and cathode is transparent so that light incident on the device may reach the bulk heterojunction layer. In some embodiments, both of the anode and cathode are transparent.

Each transparent electrode preferably has a transmittance of at least 70%, optionally at least 80%, to wavelengths in the range of 400-750 nm or 750-1000 nm or 1000-2000 nm. The transmittance may be selected according to the absorption peak of the material comprising the group of formula (I).

FIG. 1 illustrates an arrangement in which the cathode is disposed between the substrate and the anode. In other embodiments, the anode may be disposed between the cathode and the substrate.

The area of the OPD may be less than about 3 cm², less than about 2 cm², less than about 1 cm², less than about 0.75 cm², less than about 0.5 cm² or less than about 0.25 cm². The substrate may be, without limitation, a glass or plastic substrate. The substrate can be described as an inorganic semiconductor. In some embodiments, the substrate may be silicon. For example, the substrate can be a wafer of silicon. The substrate is transparent if, in use, incident light is to be transmitted through the substrate and the electrode supported by the substrate.

The bulk heterojunction layer comprises an electron donor and an electron acceptor. The bulk heterojunction layer may contain more than one electron donor and/or more than one electron acceptor. Optionally, the bulk heterojunction layer consists of the at least one electron donor and the at least one electron acceptor.

In some embodiments, the weight of the donor to the acceptor is from about 1:0.5 to about 1:2.

Preferably, the weight ratio of the donor to the acceptor is about 1:1 or about 1:1.5.

The bulk heterojunction layer may be formed by any process including, without limitation, thermal evaporation and solution deposition methods.

Preferably, the bulk heterojunction layer is formed by depositing a formulation comprising the acceptor and the electron donor dissolved or dispersed in a solvent or a mixture of two or more solvents. The formulation may be deposited by any coating or printing method including, without limitation, spin-coating, dip-coating, roll-coating, spray coating, doctor blade coating, wire bar coating, slit coating, ink jet printing, screen printing, gravure printing and flexographic printing.

The one or more solvents of the formulation may optionally comprise or consist of benzene substituted with one or more substituents selected from chlorine, C₁₋₁₀ alkyl and C₁₋₁₀ alkoxy wherein two or more substituents may be linked to form a ring is which may be unsubstituted or substituted with one or more C₁₋₆ alkyl groups, optionally toluene, xylenes, trimethylbenzenes, tetramethylbenzenes, anisole, indane and its alkyl-substituted derivatives, and tetralin and its alkyl-substituted derivatives.

The formulation may comprise a mixture of two or more solvents, preferably a mixture comprising at least one benzene substituted with one or more substituents as described above and one or more further solvents. The one or more further solvents may be selected from esters, optionally alkyl or aryl esters of alkyl or aryl carboxylic acids, optionally a C₁₋₁₀ alkyl benzoate, benzyl benzoate or dimethoxybenzene. In preferred embodiments, a mixture of trimethylbenzene and benzyl benzoate is used as the solvent. In other preferred embodiments, a mixture of trimethylbenzene and dimethoxybenzene is used as the solvent.

The formulation may comprise further components in addition to the electron acceptor, the electron donor and the one or more solvents. As examples of such components, adhesive agents, defoaming agents, deaerators, viscosity enhancers, diluents, auxiliaries, flow improvers colourants, dyes or pigments, sensitizers, stabilizers, nanoparticles, surface-active compounds, lubricating agents, wetting agents, dispersing agents and inhibitors may be mentioned.

The electron donor (p-type material) has a HOMO deeper (further from vacuum) than a LUMO of the electron acceptor (n-type material). Optionally, the gap between the HOMO level of the p-type donor material and the LUMO level of the n-type acceptor material is less than 1.4 eV.

Each of the anode and cathode may independently be a single conductive layer or may comprise a plurality of layers.

The organic photoresponsive device may comprise layers other than the anode, cathode and bulk heterojunction layer shown in FIG. 1. In some embodiments, a hole-transporting layer is disposed between the anode and the bulk heterojunction layer. In some embodiments, an electron-transporting layer is disposed between the cathode and the bulk heterojunction layer. In some embodiments, a work function modification layer is disposed between the bulk heterojunction layer and the anode, is and/or between the bulk heterojunction layer and the cathode.

In the case where the organic photoresponsive device is an organic photodetector (OPD), it may be connected to a voltage source for applying a reverse bias to the device and/or a device configured to measure photocurrent. The voltage applied to the photodetector may be variable. In some embodiments, the photodetector may be continuously biased when in use.

In some embodiments, a photodetector system comprises a plurality of photodetectors as described herein, such as an image sensor of a camera.

In some embodiments, a sensor may comprise an OPD as described herein and a light source wherein the OPD is configured to receive light emitted from the light source.

In some embodiments, the light from the light source may or may not be changed before reaching the OPD. For example, the light may be reflected, filtered, down-converted or up-converted before it reaches the OPD.

At least one of the electron donor and electron acceptor of the bulk heterojunction layer is a material comprising a group of formula (I):

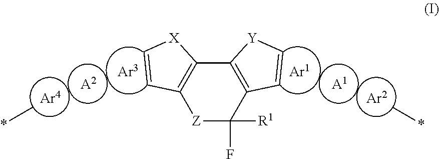

wherein:

X and Y are each independently selected from S, O or Se;

Z is O, S, NR² or CR₃²

Ar¹, Ar², Ar³ and Ar⁴ are each independently an unsubstituted or a substituted benzene, an unsubstituted or a substituted 5- or 6-membered heteroaromatic group or are absent;

A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent;

R¹ is H or a substituent;

R² is H or a substituent;

each R³ is independently H or a substituent; and

* represents a point of attachment to a hydrogen or non-hydrogen group.

Preferably, the material comprising the group of formula (I) is an electron donor of the bulk heterojunction layer, more preferably an electron donor polymer comprising a repeat unit of formula (I).

A material comprising a group of formula (I) suitable for use as an electron donor preferably has a HOMO level of at least 4.8 eV from vacuum level, optionally at least 5.0 eV, at least 5.2 eV or at least 5.4 eV from vacuum level as measured by square wave voltammetry. A shallow HOMO level allows for a small HOMO-LUMO band gap which may result in enhanced absorption at long wavelengths, e.g. more than 750 nm or more than 1000 nm. However, a material with a shallow HOMO level may be more susceptible to degradation, e.g. less stable in air, than a material having a deeper HOMO level.

HOMO and LUMO measurement by square wave voltammetry may be carried out using a CHI660D Electrochemical workstation with software (IJ Cambria Scientific Ltd), CHI 104 3 mm Glassy Carbon Disk Working Electrode (IJ Cambria Scientific Ltd), a platinum wire auxiliary electrode and a reference Electrode (Ag/AgCl) (Havard Apparatus Ltd). Acetonitrile (available as Hi-dry anhydrous grade-ROMIL) may be as the cell solution solvent. Ferrocene (available from FLUKA) may be used as the reference standard. Tetrabutylammoniumhexafluorophosphate (available from FLUKA) may be used as the cell solution salt. The HOMO and LUMO values are measured from a dilute solution (0.3 w %) in toluene in the case of a non-polymeric material or a film cast from toluene in the case of a polymer. The measurement cell contains the electrolyte, a glassy carbon working electrode, a platinum counter electrode, and a Ag/AgCl reference glass electrode. Ferrocene is added into the cell at the end of the experiment as reference material (LUMO (ferrocene)=−4.8 eV).

In the case where the material comprising a group of formula (I) is an electron donor in combination with an electron acceptor, the electron acceptor is not particularly limited and may be selected from any electron acceptor known to the skilled person. In this case, the electron acceptor may be a non-fullerene acceptor which may or may not be a material comprising a group of formula (I), or a fullerene acceptor. Non-fullerene acceptors are described in, for example, Cheng et al, “Next-generation organic photovoltaics based on non-fullerene acceptors”, Nature Photonics volume 12, pages 131-142 (2018), the contents of which are incorporated herein by reference, and which include, without limitation, PDI, ITIC, ITIC, IEICO and derivatives thereof, e.g. fluorinated derivatives thereof such as ITIC-4F and IEICO-4F. Exemplary fullerene electron acceptor materials are C₆₀, C₇₀, C₇₆, C₇₈ and C₈₄ fullerenes or a derivative thereof including, without limitation, PCBM-type fullerene derivatives (including phenyl-C61-butyric acid methyl ester (C₆₀PCBM), TCBM-type fullerene derivatives (e.g. tolyl-C61-butyric acid methyl ester (C₆₀TCBM)), and ThCBM-type fullerene derivatives (e.g. thienyl-C61-butyric acid methyl ester (C₆₀ThCBM).

In some embodiments, the material comprising the group of formula (I) is a non-polymeric compound containing at least one group of formula (I), optionally 1 or 2 groups of formula (I). Preferably, the non-polymeric compound is an electron acceptor of the bulk heterojunction layer and comprises at least one, optionally 1 or 2, electron donating groups of formula (I) and at least one electron-accepting group.

In a preferred embodiment, A¹ and A² are each independently a cyclohexane, wherein optionally one or more carbon atoms are replaced with S, NR² or O.

In the case where the material comprising a group of formula (I) is an electron acceptor in combination with an electron donor, the electron donor is not particularly limited and may be selected from any electron acceptor known to the skilled person.

Optionally, R¹ is selected from: H, F, C₁₋₂₀ alkyl wherein one or more non-adjacent, non-terminal C atoms may be replaced with O, S, COO or CO and one or more H atoms of the alkyl may be replaced with F; and phenyl which is unsubstituted or substituted with one or more substituents, optionally one or more C₁₋₁₂ alkyl groups wherein one or more non-adjacent, non-terminal C atoms may be replaced with O, S, COO or CO and one or more H atoms of the alkyl may be replaced with F.

Preferably, R¹ is F.

Optionally, R² is selected from H, C₁₋₃₀ alkyl wherein one or more non-adjacent, non-terminal C atoms may be replaced with O, S, COO or CO and one or more H atoms of the alkyl may be replaced with F; and an aromatic group Ar⁵, optionally phenyl, which is unsubstituted or substituted with one or more substituents selected from F and C₁₋₁₂ alkyl wherein one or more non-adjacent, non-terminal C atoms may be replaced with O, S, COO or CO.

Preferably, R² is selected from H, C₁₋₃₀ alkyl; unsubstituted phenyl; or phenyl substituted with one or more substituents selected from C₁₋₁₂ alkyl and C₁₋₁₂ alkoxy.

Optionally, R³ in each occurrence is independently selected from H; F; C₁₋₂₀ alkyl wherein one or more non-adjacent, non-terminal C atoms may be replaced with O, S, COO or CO and one or more H atoms of the alkyl may be replaced with F; and an aromatic group Ar⁵, optionally phenyl, which is unsubstituted or substituted with one or more substituents. Two R² groups attached to the same carbon atom may be linked to form a ring, e.g. a cycloalkyl ring or an aromatic or heteroaromatic ring, e.g. fluorene.

Substituents of Ar⁵ may be selected from selected from F; C₁₋₃₀ alkyl wherein one or more non-adjacent, non-terminal C atoms may be replaced with O, S, COO or CO; and COOH or a salt thereof.

Ar¹-Ar⁴ are preferably each benzene or thiophene, each of which is optionally and independently unsubstituted or substituted with one or more substituents.

Ar¹, Ar², Ar³, Ar⁴, A¹ and A² are each independently and optionally unsubstituted or substituted with one or more substituents, optionally one or more substituents selected from F; C₁₋₂₀ alkyl wherein one or more non-adjacent, non-terminal C atoms may be replaced with O, S, COO or CO and one or more H atoms of the alkyl may be replaced with F; and —B(R¹⁴)₂ wherein R¹⁴ in each occurrence is a substituent, optionally a C₁₋₂₀ hydrocarbyl group.

Z is preferably NR² or CR³₂.

X and Y are each preferably S.

In a preferred embodiment, Ar¹, A¹, Ar², Ar³ A² and Ar⁴ of formula (I) are absent.

In some embodiments, Ar¹ and, optionally, A¹ and Ar² are present; Ar³, Ar⁴ and A² are absent; and the group of formula (I) is a group of formula (Ia):

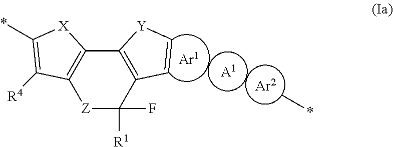

wherein R⁴ is H or a substituent.

In some embodiments, Ar^{a} and, optionally, A² and Ar⁴ are present; Ar¹, Ar^{e} and A¹ are absent; and the group of formula (I) is a group of formula (Ib):

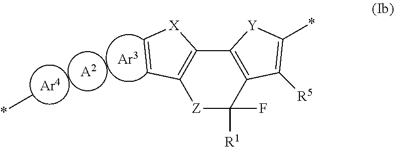

wherein R⁵ is H or a substituent.

In some embodiments, Ar¹-Ar⁴, A¹ and A² and Ar⁴ are absent; and the group of formula (I) is a group of formula (Ic):

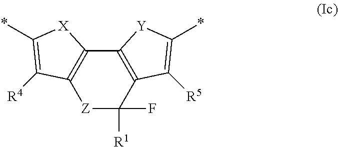

Optionally, R⁴ and R⁵ of formula (Ia), (Ib) or (Ic) are each independently selected from H; F; C₁₋₂₀ alkyl wherein one or more non-adjacent, non-terminal C atoms may be replaced with O, S, COO or CO and one or more H atoms of the alkyl may be replaced with F; and —B(R¹⁴)₂ wherein R¹⁴ in each occurrence is a substituent, optionally a C₁₋₂₀ hydrocarbyl group.

Optionally, the group of formula (I) is a group of one of the following formulae:

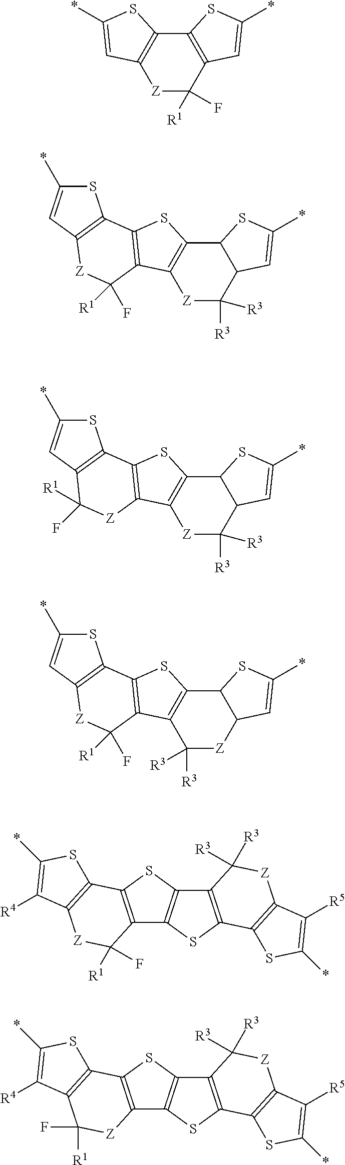

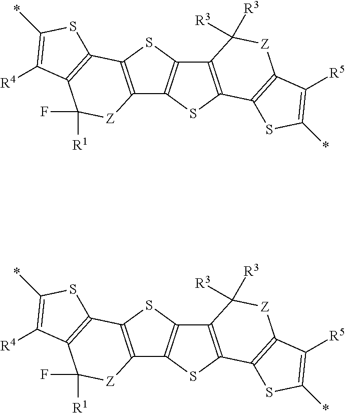

Exemplary groups of formula (I) include:

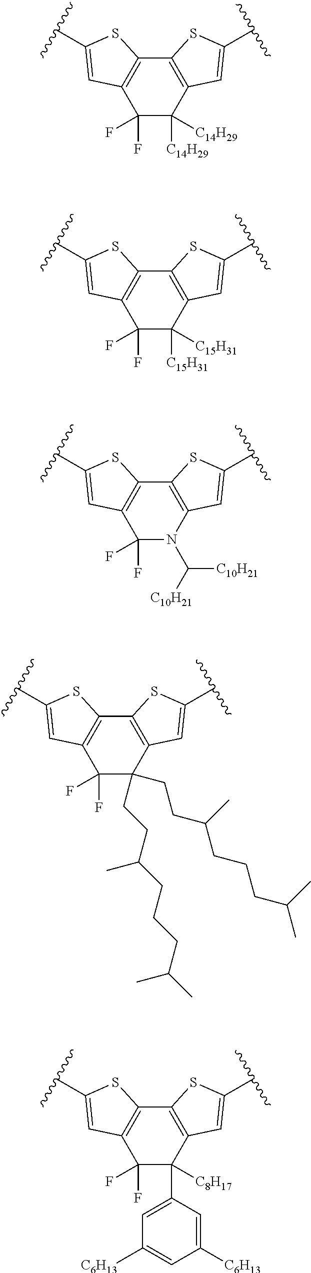

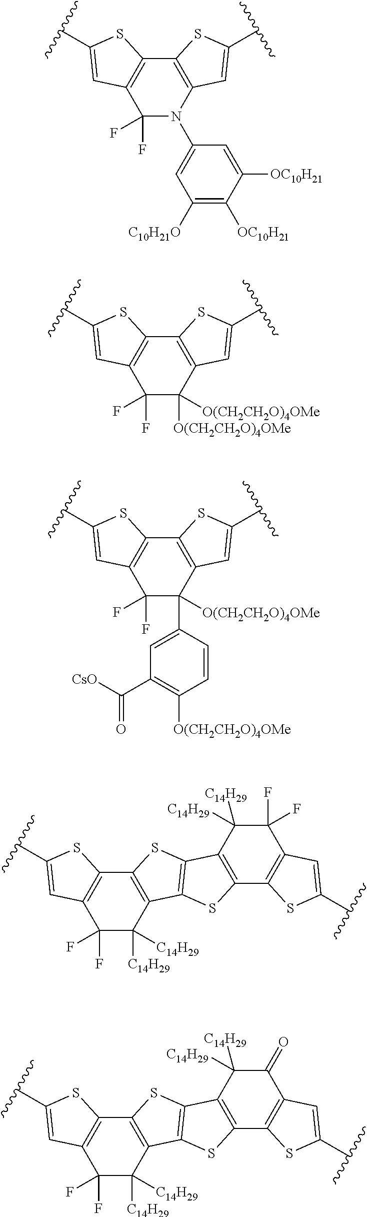

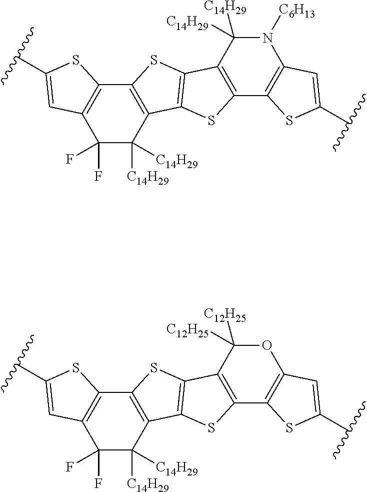

In the case where the material comprising the group of formula (I) is a polymer, the polymer comprises a repeat unit of formula (Id):

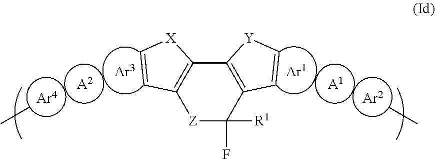

Optionally, the repeat unit of formula (Id) has formula (Ie), (If) or (Ig):

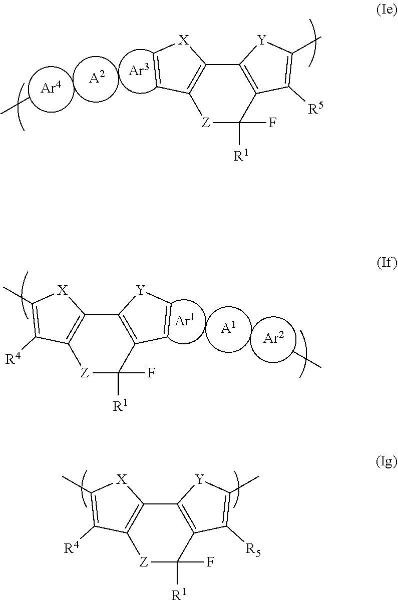

wherein X, Y, R¹ to R⁵, Ar¹ to Ar⁴, A¹ and A² are as previously defined.

The polymer is preferably a copolymer comprising electron-donating repeat units of formula (Id) and electron-accepting co-repeat units. Repeat units of formula (I) and the electron-accepting co-repeat units may together form a repeating structure in the polymer backbone of formula:

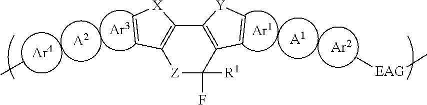

Optionally, each EAG repeat unit of the polymer (except any terminal EAG repeat unit) is adjacent to a repeat unit of formula (Id).

Optionally, each repeat unit of formula (Id) of the polymer, except any terminal repeat unit of formula (Id), is adjacent to an EAG repeat unit.

In the case where the material comprising a group of formula (I) is a non-polymeric compound, the compound preferably contains at least one electron accepting group (EAG) which may be directly or indirectly bound to the group of formula (I).

The, or each, EAG has a LUMO level that is deeper (i.e. further from vacuum) than EDG, preferably at least 1 eV deeper. The LUMO levels of EAG and EDG may be as determined by modelling the LUMO level of EAG-H or H-EAG-H with that of H-EDG-H, i.e. by replacing the bonds between EAG and EDG with bonds to a hydrogen atom. Modelling may be performed using Gaussian09 software available from Gaussian using Gaussian09 with B3LYP (functional) and LACVP* (Basis set).

Accordingly, in some embodiments, there is provided a material comprising a group of (Ih), formula (Ii), formula (Ij) or formula (Ik):

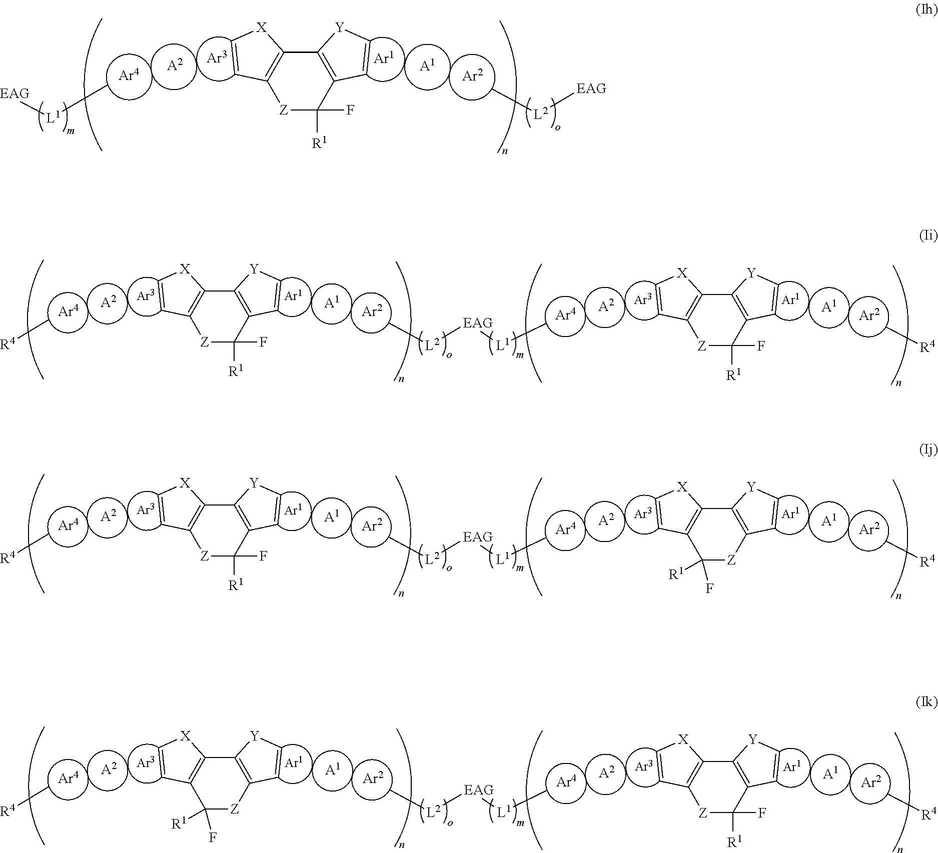

wherein:

n is an integer of 1 or more;

m and o are each independently 0 or an integer of 1 or more;

L¹ and L² each independently represent a bridging group when m and o are 1 or more or a direct bond when m and o are 0;

EAG represents an electron accepting group; and

X, Y, Z, R¹ to R⁴, Ar¹ to Ar⁴, A¹ and A² are as previously defined.

In the case where n is more than 1, e.g. 2 or 3, the groups of formula (I) may be linked in any orientation. For example, in the case where n=2, formula (I) may be any of:

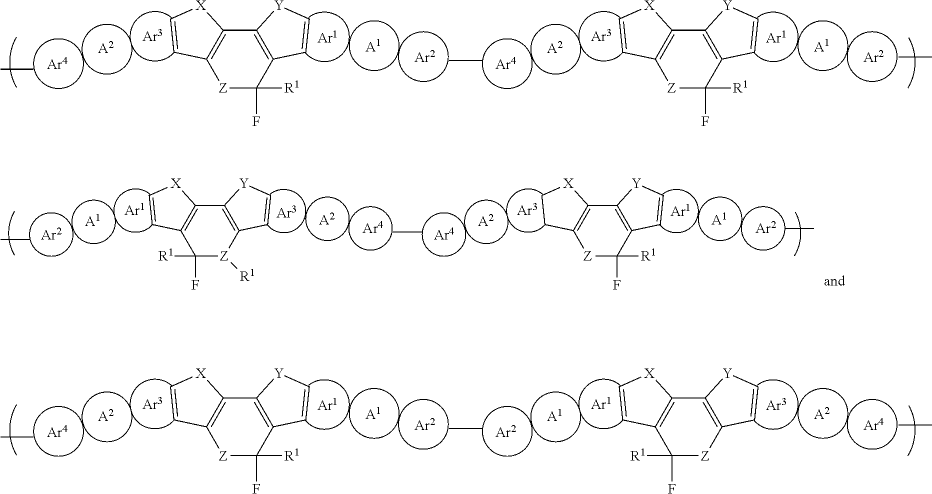

In the case where n is greater than 1, each of Ar¹-Ar⁴, R¹, A¹, A², X, Y and Z is the same or different. In some embodiments, each Z is the same. In some embodiments, one Z is one of O, S, NR² or CR³₂ and another Z is another of O, S, NR² or CR³₂.

Where the bridging groups L¹ and L² are present, L¹ and L² may each independently be a group of formula (II) or formula (III):

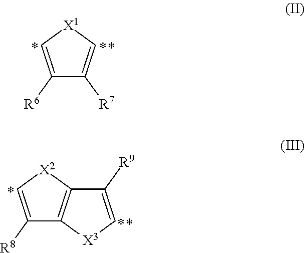

wherein:

X¹, X² and X³ are each independently S, O or Se;

* represents a point of attachment to Formula (Ih), Formula (Ii), Formula (Ij) or formula (Ik);

** represents a point of attachment to EAG; and

R⁶, R⁷, R⁸ and R⁹ are each independently H or a substituent, optionally a substituent selected from R⁴ as described above.

Preferably, L¹ and L² are each independently selected from the following formulae:

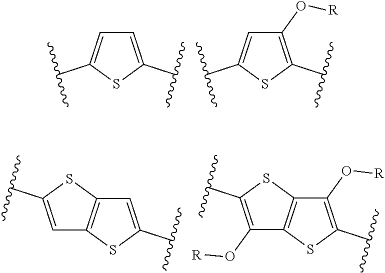

wherein R is a C₁₋₁₂ hydrocarbyl group, optionally C₁₋₁₂ alkyl.

The monovalent EAGs of formula (Ih) may be the same or different, preferably the same. Optionally, each EAG of formula (Ih) is selected from formulae (III)-(XIII):

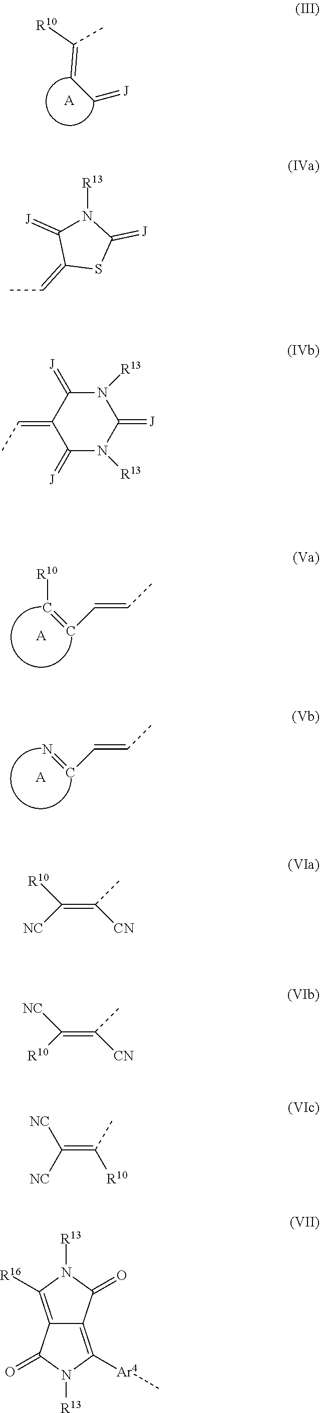

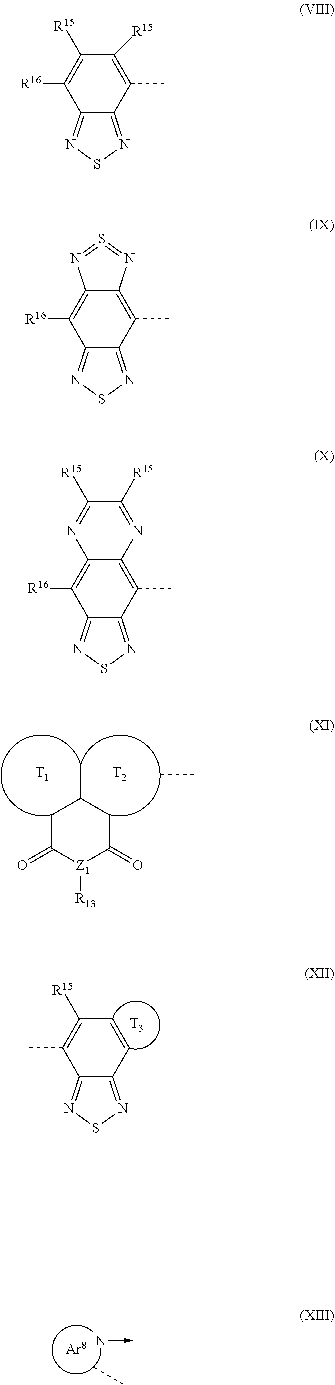

[symbol] represents a bond to L¹, L² or a position denoted by * Formula (I)

A is a 5- or 6-membered ring which is unsubstituted or substituted with one or more substituents and which may be fused to one or more further rings.

R¹⁰ is H or a substituent, preferably a substituent selected from the group consisting of C₁₋₁₂ alkyl wherein one or more non-adjacent, non-terminal C atoms may be replaced with O, S, COO or CO and one or more H atoms of the alkyl may be replaced with F; and an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO.

Preferably, R¹⁰ is H.

J is O or S.

R¹³ in each occurrence is a substituent, optionally C₁₋₁₂ alkyl wherein one or more non-adjacent, non-terminal C atoms may be replaced with O, S, COO or CO and is one or more H atoms of the alkyl may be replaced with F.

R¹⁵ in each occurrence is independently H; F; C₁₋₁₂ alkyl wherein one or more non-adjacent, non-terminal C atoms may be replaced with O, S, COO or CO and one or more H atoms of the alkyl may be replaced with F; or an aromatic group Ar², optionally phenyl, which is unsubstituted or substituted with one or more substituents selected from F and C₁₋₁₂ alkyl wherein one or more non-adjacent, non-terminal C atoms may be replaced with O, S, COO or CO.

R¹⁶ is a substituent, preferably a substituent selected from:

—(Ar³)_{w} wherein Ar³ in each occurrence is independently an unsubstituted or substituted aryl or heteroaryl group, preferably thiophene, and w is 1, 2 or 3;

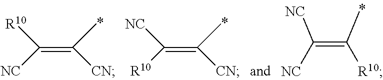

and

C₁₋₁₂ alkyl wherein one or more non-adjacent, non-terminal C atoms may be replaced with O, S, COO or CO and one or more H atoms of the alkyl may be replaced with F.

Ar⁴ is a 5-membered heteroaromatic group, preferably thiophene or furan, which is unsubstituted or substituted with one or more substituents.

Substituents of Ar³ and Ar⁴, where present, are optionally selected from C₁₋₁₂ alkyl wherein one or more non-adjacent, non-terminal C atoms may be replaced with O, S, COO or CO and one or more H atoms of the alkyl may be replaced with F.

Z¹ is N or P

T¹, T² and T³ each independently represent an aryl or a heteroaryl ring which may be fused to one or more further rings. Substituents of T¹, T² and T³, where present, are optionally selected from non-H groups of R¹⁵.

Ar⁸ is a fused heteroaromatic group which is unsubstituted or substituted with one or more non-H substituents R¹⁰.

A preferred group of formula (III) is formula (Ma).

Preferably at least one, more preferably each, EAG is a group of formula (IIIa):

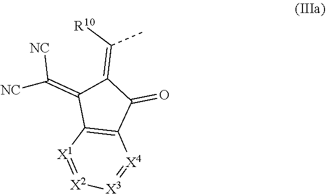

wherein:

R¹⁰ is as described above;

[symbol] represents a linking position to L¹, L² or * of formula (I); and

each X¹-X⁴ is independently CR¹² or N wherein R¹² in each occurrence is H or a substituent selected from C₁₋₂₀ hydrocarbyl and an electron withdrawing group. Optionally, the electron withdrawing group is F, Cl, Br or CN.

The C₁₋₂₀ hydrocarbyl group R¹² may be selected from C₁₋₂₀ alkyl; unsubstituted phenyl; and phenyl substituted with one or more C₁₋₁₂ alkyl groups.

Exemplary compounds of formula (IVa) or (IVb) include:

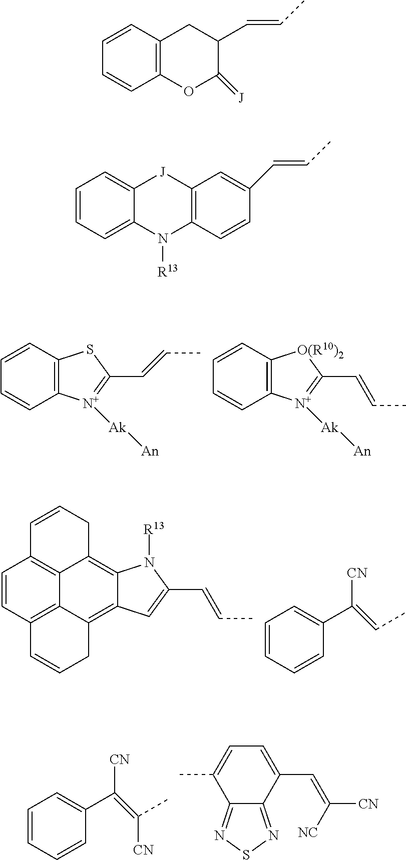

wherein Ak is a C₁₋₁₂ alkylene chain in which one or more C atoms may be replaced with O, S, CO or COO; An is an anion, optionally —SO₃⁻; and each benzene ring is independently unsubstituted or substituted with one or more substituents selected from substituents described with reference to R¹⁰.

Exemplary EAGs of formula (XI) are:

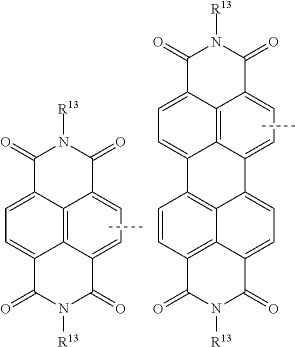

An exemplary EAG group of formula (XII) is:

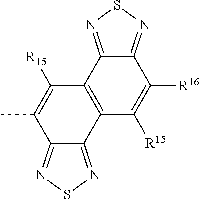

In the case where at least one EAG is a group of formula (XIII), the group of formula (I) is substituted with a group of formula —B(R¹⁴)₂ wherein R¹⁴ in each occurrence is a substituent, optionally a C₁₋₂₀ hydrocarbyl group; - - - is bound to a position denoted by * in Formula (I); and → is a bond to the boron atom of —B(R¹⁴)₂.

Optionally, R¹⁴ is selected from C₁₋₁₂ alkyl; unsubstituted phenyl; and phenyl substituted with one or more C₁₋₁₂ alkyl groups.

The group of formula (I), the group of formula (XIII) and the B(R¹⁴)₂ substituent of formula (I) may be linked together to form a 5- or 6-membered ring.

In some embodiments, EAG of formula (XIII) is selected from formulae (XIIIa), (XIIIb) and (XIIIc):

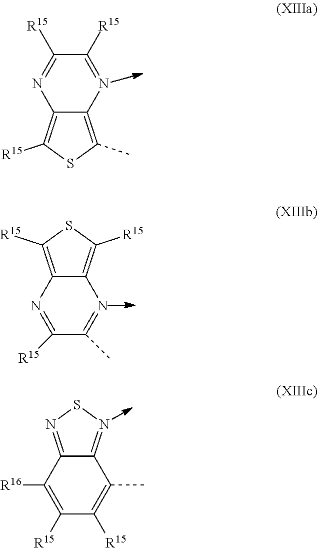

Divalent EAGs, for example of formula (Ii), (Ij) or (Ik) or EAG co-repeat units of a polymer comprising a repeat unit of formula (Id), are optionally selected from:

divalent analogues of formulae (VIII)-(X) wherein R¹⁶ is a bond to L¹, L² or * of formula (I); and

analogues (XIa) and (XIIa) of formulae (XI) and (XII), respectively:

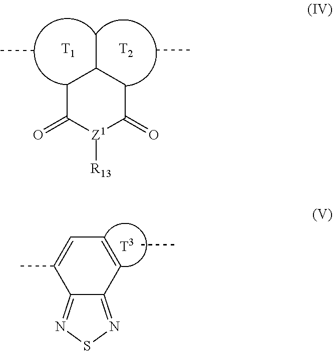

Preferable divalent EAGs, for example EAG repeat units of a polymer or EAG groups of a compound of formula (Ii), (Ij) or (Ik) are:

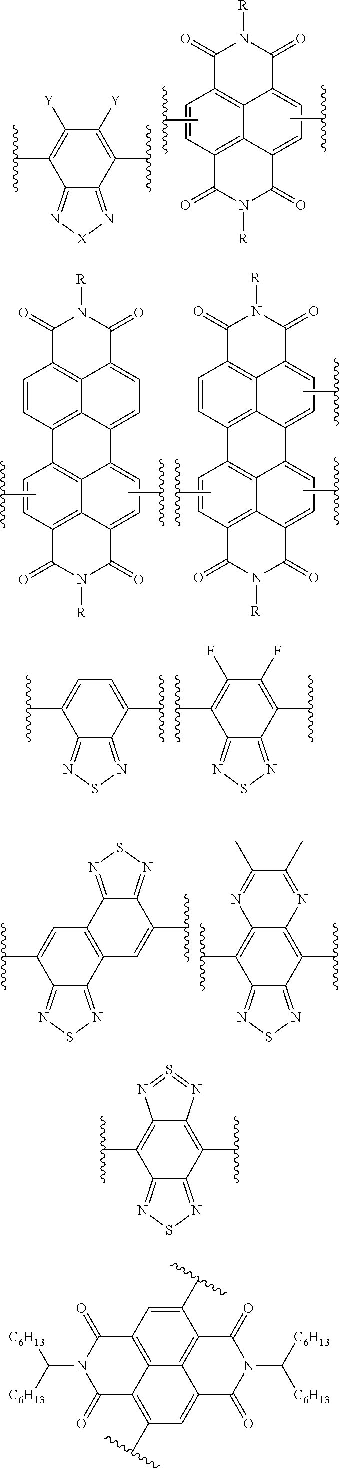

wherein Y is H or a substituent, e.g. a C₁₋₁₂ alkyl or F.

The organic photoresponsive device as described herein may be an organic photovoltaic device or an organic photodetector. An organic photodetector as described herein may be used in a wide range of applications including, without limitation, detecting the presence and/or brightness of ambient light and in a sensor comprising the organic photodetector and a light source. The photodetector may be configured such that light emitted from the light source is incident on the photodetector and changes in wavelength and/or brightness of the light may be detected, e.g. due to absorption by, reflection by and/or emission of light from an object, e.g. a target material in a sample disposed in a light path between the light source and the organic photodetector. The sample may be a non-biological sample, e.g. a water sample, or a biological sample taken from a human or animal subject. The sensor may be, without limitation, a gas sensor, a biosensor, an X-ray imaging device, an image sensor such as a camera image sensor, a motion sensor (for example for use in security applications) a proximity sensor or a fingerprint sensor. A 1D or 2D photosensor array may comprise a plurality of photodetectors as described herein in an image sensor.

### Examples

Synthesis

A group of formula (I) in which Z is CR³₂ may be prepared according to Scheme 1:

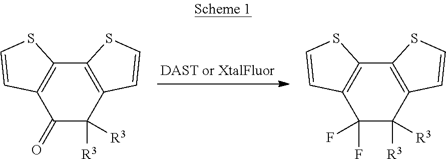

A group of formula (I) in which Z is NR² may be prepared according to Scheme 2:

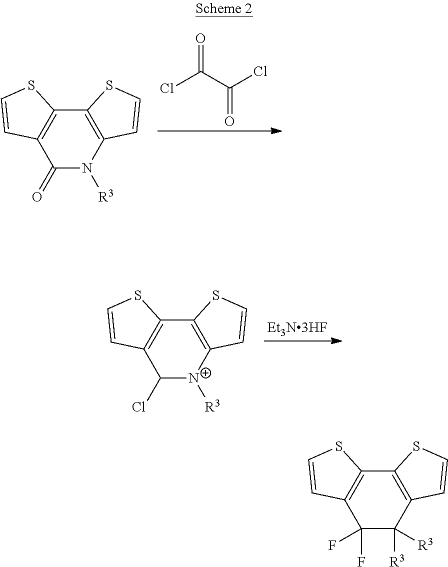

Modelling Data

HOMO and LUMO levels of compounds of formula H D A D A D H were modelled in which H is hydrogen; D is an electron donor as shown in Table 1; and A is an electron acceptor as shown in Table 1.

Quantum chemical modelling was performed using Gaussian09 software available from Gaussian using Gaussian09 with B3LYP (functional) and LACVP* (Basis set).

TABLE 1

| | | | HOMO/ | LUMO/ | Eg/ |
|---|---|---|---|---|---|
| Material | Donor D | Acceptor A | eV | eV | eV |
| Comparative Compound 1A | 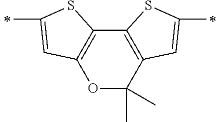 | 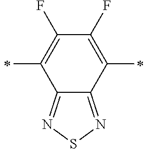 | −4.654 | −2.923 | 1.730 |
| Comparative Compound 1B | 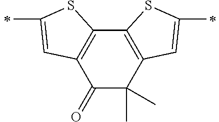 | 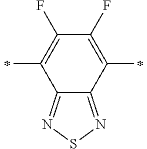 | −5.222 | −3.221 | 2.001 |
| Comparative Compound 1C | 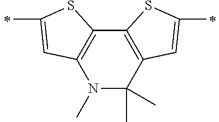 | 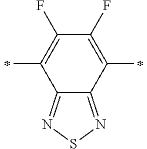 | −4.607 | −2.822 | 1.784 |
| Compound Example 1A | 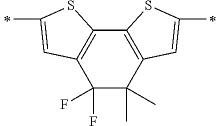 | 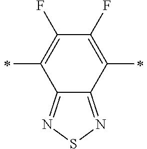 | −5.064 | −3.118 | 1.946 |
| Compound Example 1B | 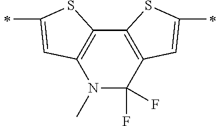 | 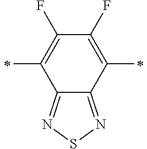 | −4.918 | −3.091 | 1.828 |
| Comparative Compound 2A | 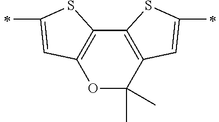 | 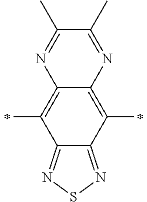 | −4.245 | −3.205 | 1.041 |
| Compound Example 2A | 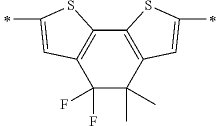 | 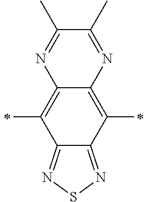 | −4.617 | −3.378 | 1.239 |

1. A material comprising a group of formula (I): 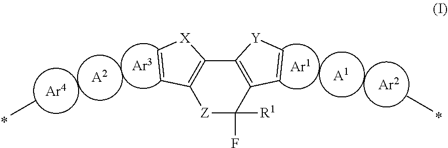
wherein:
X and Y are each independently selected from S, O or Se;
Z is O, S, NR² or CR³₂
Ar¹, Ar², Ar³ and Ar⁴ are each independently an unsubstituted or a substituted benzene, an unsubstituted or a substituted 5- or 6-membered heteroaromatic group or are absent;
A¹ and A² are each independently an unsubstituted or a substituted benzene, an unsubstituted or a substituted 5- or 6-membered heteroaromatic group, a non-aromatic 6-membered ring having ring atoms selected from C, N, S and O or are absent;
R¹ is H or a substituent;
R² is H or a substituent;
each R³ is independently H or a substituent; and
represents a point of attachment to a hydrogen or non-hydrogen group.

2. The material according to claim 1, wherein Ar¹, A¹, Ar², Ar³ A² and Ar⁴ are absent and the group of formula (I) has formula (Ic):

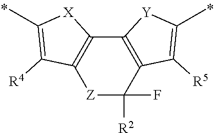

3. The material according to claim 1 wherein the group of formula (I) is an electron donor group, the material further comprising at least one electron-accepting group bound directly to the group of formula (I).

4. The material according to claim 1, wherein the material is a polymer comprising a repeat unit of formula (Id):

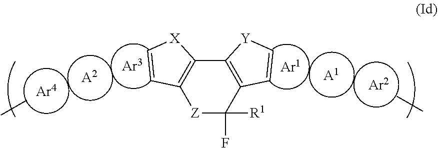

5. The material as claimed in claim 3, wherein the repeat unit of formula (Id) is selected from repeat units of formulae (Ie), (If) and (Ig):

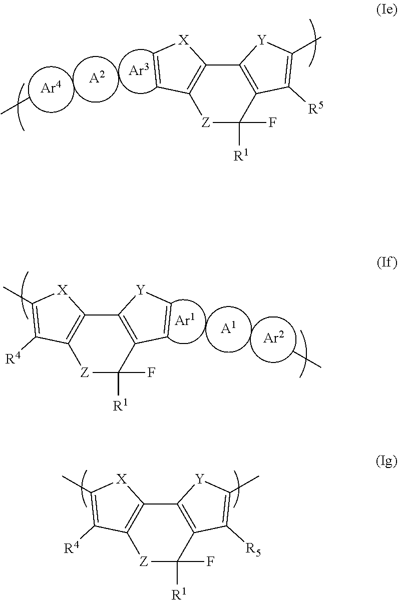

6. The material according to claim 4 wherein the repeat unit of formula (I) is an electron-donating repeat unit and wherein the polymer further comprises an electron accepting co-repeat unit.

7. The material according to claim 6 wherein the polymer comprises a repeating structure of formula:

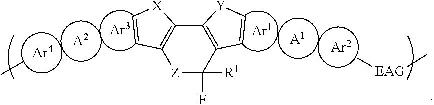

8. A composition comprising an electron donor material and an electron acceptor material wherein the electron donor material is the material comprising a group of formula (I) according to claim 1.

9. A composition comprising an electron donor material and an electron acceptor material wherein the electron acceptor material is the material comprising a group of formula (I) according to claim 1.

10. A formulation comprising one or more solvents and a material according to claim 1 wherein the material comprising the group of formula (I) is dissolved or dispersed in the one or more solvents.

11. The formulation according to claim 10 wherein the material comprising a group of formula (I) is one of an electron donor material and an electron acceptor material and the formulation further comprises the other of an electron donor material and an electron acceptor material.

12. A photoresponsive device comprising an anode, a cathode and a photosensitive layer disposed between the anode and the cathode, wherein the photosensitive layer comprises a material comprising a group of formula (I) according to claim 1.

13. The photoresponsive device according to claim 12 wherein the material comprising a group of formula (I) is one of an electron donor material and an electron acceptor material and the photosensitive layer further comprises the other of an electron donor material and an electron acceptor material.

14. The photoresponsive device as claimed in claim 12, wherein the photoresponsive device is an organic photodetector.

15. A photosensor comprising a light source and a photoresponsive device as claimed in claim 14, wherein the photoresponsive device is configured to detect light emitted from the light source.

16. The photosensor according to claim 15, wherein the light source emits light having a peak wavelength greater than 750 nm.

17. The photosensor according to claim 15 configured to receive a sample in a light path between the organic photodetector and the light source.

18. A method of forming an organic photoresponsive device according to claim 12 comprising formation of the photosensitive organic layer over one of the anode and cathode and formation of the other of the anode and cathode over the photosensitive organic layer.

19. A method according to claim 16 wherein formation of the photosensitive organic layer comprises deposition of a formulation comprising one or more solvents and the material comprising the group of formula (I) dissolved or dispersed in the one or more solvents.

20. A method of determining the presence and/or concentration of a target material in a sample, the method comprising illuminating the sample and measuring a response of a photoresponsive device as claimed in claim 13.
